(19) United States
(12) Patent Application Publication
(10) Pub. No.: US 2024/0350787 A1
(43) Pub. Date: Oct. 24, 2024

(54) SEALING CAP WITH ADJUSTABLE APERTURE AND ENDOSCOPE MASK

(71) Applicant: MEDPLUS INC., Guangzhou (CN)

(72) Inventors: Peibo LUO, Guangzhou (CN); Mingde LIAO, Guangzhou (CN); Zhentui CHEN, Guangzhou (CN)

(73) Assignee: MEDPLUS INC., Guangzhou (CN)

(21) Appl. No.: 18/635,054

(22) Filed: Apr. 15, 2024

(30) Foreign Application Priority Data
Apr. 23, 2023 (CN) 202320936094.8

(51) Int. Cl. A61M 39/00

(52) U.S. Cl. CPC A61M 39/00; A61M 2039/0009; A61M 2039/0072

(57) ABSTRACT

A sealing cap with adjustable aperture and an endoscope mask, with the key technical solution being: the endoscope mask includes a mask body and a sealing cap with adjustable aperture; the sealing cap with adjustable aperture comprises: a fixing seat; a cover body, which is detachably mounted on the fixing seat; a sealing component, used to adapt to endoscopes of different specifications; one end of the sealing component is set on the cover body, and the other end of the sealing component is connected to the fixing seat. The invention has the advantage of allowing the mask to adapt to endoscopes of different specifications to improve work efficiency.

### CROSS REFERENCE TO RELATED APPLICATIONS

This application is based upon and claims foreign priority to Chinese Patent Application No. CN202320936094.8, filed on Apr. 23, 2023, the entire contents of which are incorporated herein by reference.

### TECHNICAL FIELD

The invention pertains to the field of medical device technology, more specifically, it relates to a sealing cap with adjustable aperture and an endoscope mask.

### BACKGROUND

Endoscope masks are used in scenarios such as gastroscopy and fiberoptic bronchoscopy examinations, and can provide ventilation to the patient during the examination through side openings for oxygen or anesthetic gases, allowing for simultaneous intubation and ventilation. However, the seal between the mask and the endoscope must be considered.

Clinically, due to the various examination needs of different patients, endoscopes also come in various diameters. However, current masks on the market can only fit a single specification of endoscope, requiring doctors to consider the specification of the mask to be used before surgery to fit different endoscopes, which brings various inconveniences to clinical doctors and lowers work efficiency, thus improvement is needed.

### SUMMARY

To address the deficiencies in the existing technology, the objective of this invention is to provide a sealing cap with adjustable aperture and endoscope mask, which has the advantage of allowing the mask to adapt to endoscopes of different specifications to improve work efficiency.

The first technical objective of this invention is achieved through the following technical solution: a sealing cap with adjustable aperture, including: a fixing seat; a cover body, which is detachably mounted on the fixing seat; a sealing component, used to adapt to endoscopes of different specifications; one end of the sealing component is set on the cover body, and the other end of the sealing component is connected to the fixing seat.

In one embodiment of the above technical solution, the sealing component includes: a membrane, and a rotating inner sleeve and a rotating outer sleeve for driving the membrane; one end of the rotating inner sleeve is rotatably mounted on the cover body, and the other end of the rotating inner sleeve is connected to the rotating outer sleeve; one end of the membrane is connected to the rotating outer sleeve, and the other end of the membrane is connected to the fixing seat.

In one embodiment of the above technical solution, both the rotating inner sleeve and the rotating outer sleeve are annular sleeves; the upper end of the membrane is fixed between the rotating inner sleeve and the rotating outer sleeve.

In one embodiment of the above technical solution, both the rotating inner sleeve and the rotating outer sleeve are plastic circular ring sleeves.

In one embodiment of the above technical solution, a protruding ring is set at the lower end of the membrane; a mounting groove that can accommodate the protruding ring is set on the fixing seat.

In one embodiment of the above technical solution, a protruding platform that can clamp the endoscope mask corresponding to the cover body is set at the lower end of the fixing seat.

In one embodiment of the above technical solution, a cushioning pad is set at the upper end of the protruding platform.

In one embodiment of the above technical solution, the cushioning pad is made of silicone pad and/or rubber pad.

The second technical objective of this invention is achieved through the following technical solution: an endoscope mask, including: a mask body; it also includes a sealing cap with adjustable aperture.

Optionally, a mounting platform is set on the outside of the mask body; one end of the mounting platform abuts the cushioning pad, and the other end of the mounting platform abuts the cover body.

In summary, this invention has the following beneficial effects:

1. The sealing cap with adjustable aperture of this application, with both ends of the sealing component respectively connected to the fixing seat and the cover body, and the sealing component internally equipped with a membrane and rotating inner and outer sleeves that can drive the rotation of the membrane. When the rotating sleeves are turned, since one end of the membrane is fixed and the other end rotates, a hole that can contract and clamp the endoscope is formed in the middle of the membrane. As the rotation angle of the rotating sleeves changes, the aperture of the hole formed by the membrane also changes correspondingly, thus adapting to endoscopes of different specifications through different apertures.

2. After using the sealing cap with adjustable aperture of this application, only one mask body is needed to be suitable for endoscopes of various different specifications, which is convenient for clinical doctors to adjust, thereby improving the overall work efficiency.

### BRIEF DESCRIPTION OF THE DRAWINGS

FIG. 1 is a cross-sectional schematic diagram of the sealing cap with adjustable aperture in this invention;

FIG. 2 is a partial schematic diagram of FIG. 1;

FIG. 3 is a cross-sectional schematic diagram of the mask body in this invention;

FIG. 4 is a partial schematic diagram of FIG. 3;

FIG. 5 is a schematic diagram of the overall structure of this invention.

List of reference numerals: 1, fixing seat; 2, cover body; 3, sealing component; 31, membrane; 32, rotating inner sleeve; 33, rotating outer sleeve; 41, protruding ring; 42, mounting groove; 51, protruding platform; 52, cushioning pad; 6, mask body; 7, mounting platform.

### DETAILED DESCRIPTION OF THE EMBODIMENTS

To make the objectives, features, and advantages of this invention more clear and understandable, the specific embodiments of this invention are described in detail below in conjunction with the accompanying drawings. Several embodiments of this invention are given in the drawings. However, this invention can be implemented in many different forms and should not be construed as limited to the embodiments set forth herein.

In this invention, unless otherwise explicitly stated and limited, terms such as “installation,” “connection,” “fixation,” and similar terms should be understood broadly, for example, they can be fixed connections or detachable connections, or integrally formed connections; they can be mechanical connections or electrical connections; they can be directly connected or indirectly connected through an intermediary medium, and can be the internal communication of two components. For those skilled in the art, the specific meanings of the above terms in this invention can be understood according to specific circumstances. The terms “first,” “second” are used for descriptive purposes only and cannot be understood to indicate or imply relative importance or implicitly indicate the quantity of the indicated technical features. Thus, features defined with “first,” “second” can explicitly or implicitly include one or more of such features.

In this invention, unless otherwise explicitly stated and limited, the first feature “above” or “below” the second feature can include the first and second features directly contacting each other, or the first and second features not directly contacting each other but through another feature between them. Moreover, the first feature “above,” “above,” and “on top of” the second feature includes the first feature being directly above or obliquely above the second feature, or merely indicating that the first feature is higher than the second feature in terms of horizontal level. The first feature “below,” “below,” and “underneath” the second feature includes the first feature being directly below or obliquely below the second feature, or merely indicating that the first feature is lower than the second feature in terms of horizontal level. The terms “vertical,” “horizontal,” “left,” “right,” “up,” “down,” and similar expressions are used for illustrative purposes only and are not meant to indicate or imply that the indicated devices or components must have a specific orientation, be constructed and operated in a specific orientation, and thus should not be understood as limiting this invention.

The following describes this invention in detail with reference to the drawings and embodiments.

### Embodiment 1

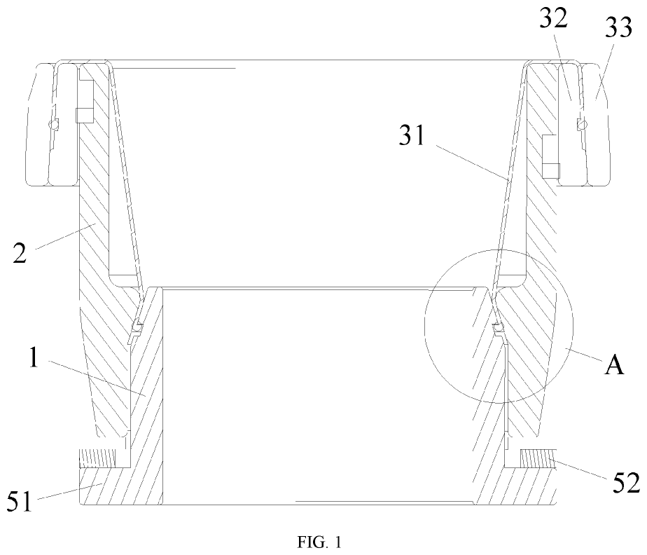

As shown in FIGS. 1 to 4, this invention provides a sealing cap with adjustable aperture, including: a fixing seat 1; a cover body 2; the cover body 2 is detachably mounted on the fixing seat 1; a sealing component 3, used to adapt to endoscopes of different specifications; one end of the sealing component 3 is set on the cover body 2, and the other end of the sealing component 3 is connected to the fixing seat 1.

In this embodiment, the cover body 2 is detachably mounted on the fixing seat 1, and after the fixing seat 1 is combined with the cover body 2, it can be connected to the endoscope mask; both ends of the sealing component 3 are respectively connected to the fixing seat 1 and the cover body 2, and when the sealing component 3 works, it can lock endoscopes of different specifications, thus only one endoscope mask is needed, and through the sealing cap, it can be used for endoscopes of various different specifications, which is convenient for clinical doctors to adjust, thereby improving the overall work efficiency.

Further, the sealing component 3 includes: a membrane 31, and a rotating inner sleeve 32 and a rotating outer sleeve 33 for driving the membrane 31; one end of the rotating inner sleeve 32 is rotatably mounted on the cover body 2, and the other end of the rotating inner sleeve 32 is connected to the rotating outer sleeve 33; one end of the membrane 31 is connected to the rotating outer sleeve 33, and the other end of the membrane 31 is connected to the fixing seat 1. In this embodiment, the rotating inner sleeve 32 is rotatably mounted on the upper end of the cover body 2, the rotating outer sleeve 33 is installed on the outside of the rotating inner sleeve 32, one end of the membrane 31 is fixed on the rotating outer sleeve 33, and the other end is fixed on the fixing seat 1, when the rotating inner sleeve 32 and the rotating outer sleeve 33 rotate, they can drive the upper end of the membrane 31 to rotate together;

When the rotating sleeves are turned, since one end of the membrane 31 is fixed and the other end rotates, the middle part of the membrane 31 forms a hole that can contract and clamp the endoscope, and as the rotation angle of the rotating sleeves changes, the aperture of the hole formed by the membrane 31 also changes correspondingly, making the hole have the following states:

\textcircled{1} In the initial state, the hole is at its largest; \textcircled{2} When the rotating sleeves are turned about 30°, the aperture of the hole is 29 mm; \textcircled{3} When the rotating sleeves are turned about 90°, the aperture of the hole is 15 mm; \textcircled{4} When the rotating sleeves are turned about 120°, the aperture of the hole is 10 mm; \textcircled{5} When the rotating sleeves are turned about 160°, the aperture of the hole is 5 mm; \textcircled{6} When the rotating sleeves are turned about 180°, the hole is completely closed;

The greater the rotation angle of the rotating sleeves, the smaller the aperture of the hole formed, through the above operation method, the membrane 31 can form holes of different apertures, thus allowing the sealing component 3 to adapt to endoscopes of different specifications with different apertures, which is convenient to operate, simple in structure, and highly practical.

Further, both the rotating inner sleeve 32 and the rotating outer sleeve 33 are annular sleeves; the upper end of the membrane 31 is fixed between the rotating inner sleeve 32 and the rotating outer sleeve 33.

In this embodiment, both the rotating inner sleeve 32 and the rotating outer sleeve 33 are annular sleeves, using an annular structure, making the rotating sleeves drive the membrane 31 to rotate around the cover body 2 more smoothly; the upper end of the membrane 31 is fixed between the rotating inner sleeve 32 and the rotating outer sleeve 33, both of which can clamp the membrane 31.

Further, both the rotating inner sleeve 32 and the rotating outer sleeve 33 are plastic circular ring sleeves.

In this embodiment, both the rotating inner sleeve 32 and the rotating outer sleeve 33 use plastic circular ring sleeves, the production cost of plastic circular ring sleeves is low, and they are lightweight, suitable for use in medical devices.

Further, a protruding ring 41 is set at the lower end of the membrane 31; a mounting groove 42 that can accommodate the protruding ring 41 is set on the fixing seat 1.

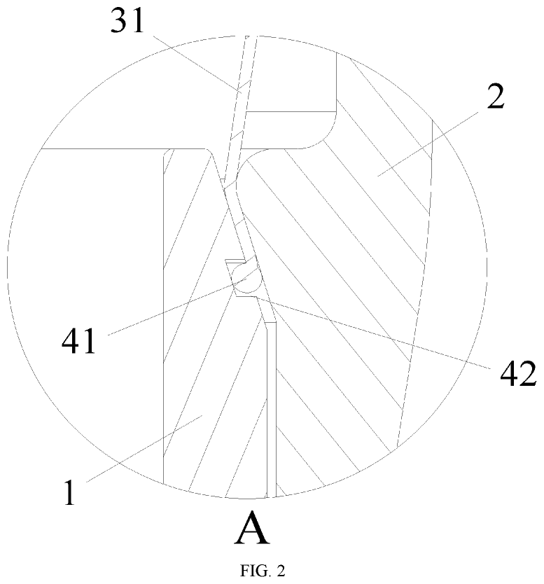

In this embodiment, a protruding ring 41 is set at the lower end of the membrane 31, and a mounting groove 42 is set on the fixing seat 1, after the protruding ring 41 is installed into the mounting groove 42, it can be fixed on the fixing seat 1 by the cover body 2 that closely fits on the outside (see FIG. 2), thus preventing the membrane 31 from slipping or misaligning during work, making the fixation of the lower end of the membrane 31 more stable.

Further, a protruding platform 51 that can clamp the endoscope mask corresponding to the cover body 2 is set at the lower end of the fixing seat 1.

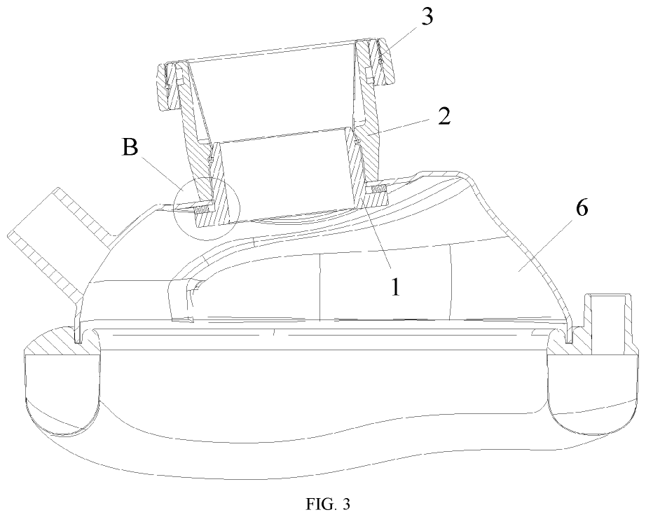

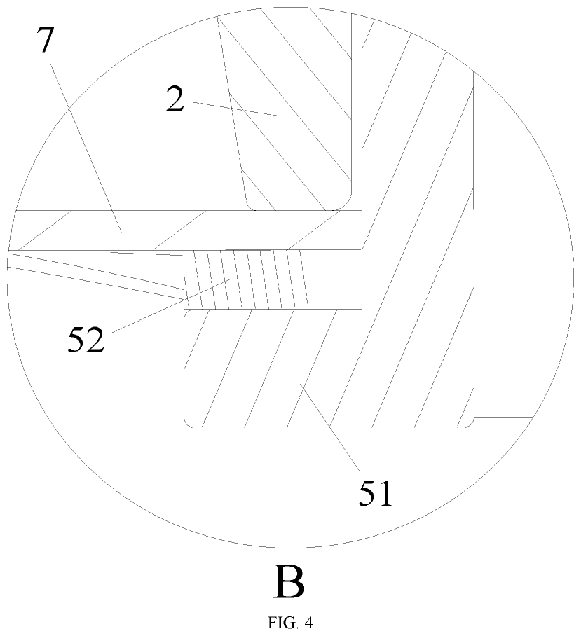

In this embodiment, the lower end of the fixing seat 1 is circumferentially provided with a protruding platform 51, when the cover body 2 is installed on the fixing seat 1, a clamping structure that can clamp the external endoscope mask is formed between the cover body 2 and the protruding platform 51 (see FIGS. 3 and 4), thus allowing the sealing cap to be installed on the endoscope mask as a whole.

Further, a cushioning pad 52 is set at the upper end of the protruding platform 51. The cushioning pad 52 is made of silicone pad and/or rubber pad.

In this embodiment, a cushioning pad 52 is set at the upper end of the protruding platform 51, the cushioning pad 52 can be made of silicone pad or rubber pad, the cushioning pad 52 can play a buffering role when the cover body 2 and the protruding platform 51 clamp the endoscope mask, not only preventing damage to the endoscope mask during clamping but also making the connection between the fixing seat 1 and the endoscope mask more stable. Additionally, it can fill the gap between the protruding platform 51 and the mask, ensuring a sealing effect between the sealing cap and the endoscope mask, thus maintaining the device's sealing performance.

### Embodiment 2

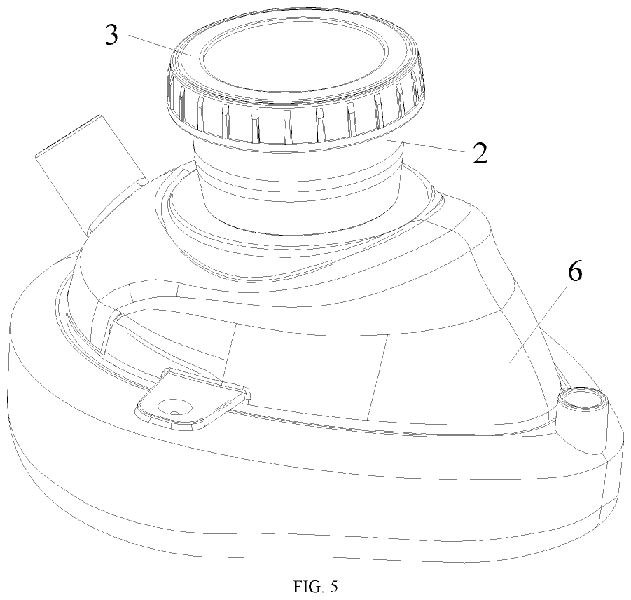

As shown in FIGS. 3 to 5, this embodiment provides an endoscope mask, including: a mask body 6; it also includes a sealing cap with adjustable aperture. Optionally, a mounting platform 7 is set on the outside of the mask body 6; one end of the mounting platform 7 abuts the cushioning pad 52, and the other end of the mounting platform 7 abuts the cover body 2.

In this embodiment, the sealing cap is fixed on the mask body 6 through the clamping structure formed between the fixing seat 1 and the cover body 2, with a mounting platform 7 set on the outside of the mask body 6, one end of the mounting platform 7 abutting the cushioning pad 52, and the other end abutting the cover body 2 (see FIG. 4), thus allowing the aforementioned clamping structure to be fixed on the mounting platform 7, achieving a stable connection between the mask body 6 and the sealing cap with adjustable aperture;

Furthermore, a positioning pin structure (such as a protrusion and groove positioning structure) is also set between the fixing seat 1 and the mounting platform 7, which can position the fixing seat 1 on the mounting platform 7, preventing the fixing seat 1 from rotating together with the sealing component during its rotation, thereby ensuring that the fixing seat 1 part remains in a stable fixed state while the sealing component is working.

This invention of a sealing cap with adjustable aperture and endoscope mask has the advantage of allowing the mask to adapt to endoscopes of different specifications to improve work efficiency.

The above-described embodiments are only the preferred embodiments of this invention, and the scope of protection of this invention is not limited to the above embodiments. Any technical solution under the inventive concept of this invention belongs to the protection scope of this invention. It should be noted that for those skilled in the art, several improvements and embellishments made without departing from the principles of this invention should also be considered as the protection scope of this invention.

1. A sealing cap with adjustable aperture, comprising:
a fixing seat;
a cover body, which is detachably mounted on the fixing seat; and
a sealing component, used to adapt to endoscopes of different specifications; one end of the sealing component is set on the cover body, and the other end of the sealing component is connected to the fixing seat.

2. The sealing cap with adjustable aperture according to claim 1, wherein the sealing component includes: a membrane, a rotating inner sleeve, and a rotating outer sleeve for driving the membrane; one end of the rotating inner sleeve is rotatably mounted on the cover body, and the other end of the rotating inner sleeve is connected to the rotating outer sleeve; one end of the membrane is connected to the rotating outer sleeve, and the other end of the membrane is connected to the fixing seat.

3. The sealing cap with adjustable aperture according to claim 2, wherein both the rotating inner sleeve and the rotating outer sleeve are annular sleeves; and
the upper end of the membrane is fixed between the rotating inner sleeve and the rotating outer sleeve.

4. The sealing cap with adjustable aperture according to claim 3, wherein both the rotating inner sleeve and the rotating outer sleeve are plastic circular ring sleeves.

5. The sealing cap with adjustable aperture according to claim 4, wherein a protruding ring is set at the lower end of the membrane; and
a mounting groove that accommodates the protruding ring is set on the fixing seat.

6. The sealing cap with adjustable aperture according to claim 1, wherein a protruding platform, which is adapted to clamp the endoscope mask in correspondence with the cover body, is set at the lower end of the fixing seat.

7. The sealing cap with adjustable aperture according to claim 6, wherein a cushioning pad is set at the upper end of the protruding platform.

8. The sealing cap with adjustable aperture according to claim 7, wherein the cushioning pad is a silicone pad and/or a rubber pad.

9. An endoscope mask, comprising: a mask body; wherein the mask body further comprises the sealing cap with adjustable aperture according to claim 1.

10. The endoscope mask according to claim 9, wherein a mounting platform is set on the outside of the mask body; one end of the mounting platform abuts the cushioning pad, and the other end of the mounting platform abuts the cover body.

11. The sealing cap with adjustable aperture according to claim 2, wherein a protruding platform, which is adapted to clamp the endoscope mask in correspondence with the cover body, is set at the lower end of the fixing seat.

12. The sealing cap with adjustable aperture according to claim 3, wherein a protruding platform, which is adapted to clamp the endoscope mask in correspondence with the cover body, is set at the lower end of the fixing seat.

13. The sealing cap with adjustable aperture according to claim 4, wherein a protruding platform, which is adapted to clamp the endoscope mask in correspondence with the cover body, is set at the lower end of the fixing seat.

14. The sealing cap with adjustable aperture according to claim 5, wherein a protruding platform, which is adapted to clamp the endoscope mask in correspondence with the cover body, is set at the lower end of the fixing seat.

15. An endoscope mask, comprising: a mask body; wherein the mask body further comprises the sealing cap with adjustable aperture according to claim 2.

16. An endoscope mask, comprising: a mask body; wherein the mask body further comprises the sealing cap with adjustable aperture according to claim 3.

17. An endoscope mask, comprising: a mask body; wherein the mask body further comprises the sealing cap with adjustable aperture according to claim 4.

18. An endoscope mask, comprising: a mask body; wherein the mask body further comprises the sealing cap with adjustable aperture according to claim 5.

19. An endoscope mask, comprising: a mask body; wherein the mask body further comprises the sealing cap with adjustable aperture according to claim 6.

20. An endoscope mask, comprising: a mask body; wherein the mask body further comprises the sealing cap with adjustable aperture according to claim 7.
